(19) United States
(12) Patent Application Publication
(10) Pub. No.: US 2024/0099706 A1
(43) Pub. Date: Mar. 28, 2024

(54) ADAPTOR FOR TORQUER

(71) Applicant: Corindus, Inc., Newton, MA (US)

(72) Inventor: Christopher Zirps, Sharon, MA (US)

(21) Appl. No.: 18/257,401

(22) PCT Filed: Jan. 14, 2021
(86) PCT No.: PCT/US2021/070033

§ 371 Date: Jun. 14, 2023

(51) Int. Cl. A61B 17/00; A61B 34/30

(52) U.S. Cl. CPC A61B 17/00234; A61B 34/30; A61B 2017/00292; A61B 2017/00486; A61B 2034/301; A61M 25/09041

(57) ABSTRACT

An adaptor engaging a torquer includes an adaptor body having a pathway therethrough. The adaptor body includes a receptacle portion defining a cavity having an opening at a first end of the adaptor body. The torquer is received within the cavity through the opening.

### BACKGROUND

Catheters and other elongated medical devices (EMDs) may be used for minimally invasive medical procedures for the diagnosis and treatment of diseases of various vascular systems, including neurovascular intervention (NVI) also known as neurointerventional surgery, percutaneous coronary intervention (PCI) and peripheral vascular intervention (PVI). These procedures typically involve navigating a guidewire through the vasculature, and via the guidewire advancing a catheter to deliver therapy. The catheterization procedure starts by gaining access into the appropriate vessel, such as an artery or vein, with an introducer sheath using standard percutaneous techniques. Through the introducer sheath, a sheath or guide catheter is then advanced over a diagnostic guidewire to a primary location such as an internal carotid artery for NVI, a coronary ostium for PCI, or a superficial femoral artery for PVI. A guidewire suitable for the vasculature is then navigated through the sheath or guide catheter to a target location in the vasculature. In certain situations, such as in tortuous anatomy, a support catheter or microcatheter is inserted over the guidewire to assist in navigating the guidewire. The physician or operator may use an imaging system (e.g., fluoroscope) to obtain a cine with a contrast injection and select a fixed frame for use as a roadmap to navigate the guidewire or catheter to the target location, for example, a lesion. Contrast-enhanced images are also obtained while the physician delivers the guidewire or catheter so that the physician can verify that the device is moving along the correct path to the target location. While observing the anatomy using fluoroscopy, the physician manipulates the proximal end of the guidewire or catheter to direct the distal tip into the appropriate vessels toward the lesion or target anatomical location and avoid advancing into side branches.

Robotic catheter-based procedure systems have been developed that may be used to aid a physician in performing catheterization procedures such as, for example, NVI, PCI and PVI. Examples of NVI procedures include coil embolization of aneurysms, liquid embolization of arteriovenous malformations and mechanical thrombectomy of large vessel occlusions in the setting of acute ischemic stroke. In an NVI procedure, the physician uses a robotic system to gain target lesion access by controlling the manipulation of a neurovascular guidewire and microcatheter to deliver the therapy to restore normal blood flow. Target access is enabled by the sheath or guide catheter but may also require an intermediate catheter for more distal territory or to provide adequate support for the microcatheter and guidewire. The distal tip of a guidewire is navigated into, or past, the lesion depending on the type of lesion and treatment. For treating aneurysms, the microcatheter is advanced into the lesion and the guidewire is removed and several embolization coils are deployed into the aneurysm through the microcatheter and used to block blood flow into the aneurysm. For treating arteriovenous malformations, a liquid embolic is injected into the malformation via a microcatheter. Mechanical thrombectomy to treat vessel occlusions can be achieved either through aspiration and/or use of a stent retriever. Depending on the location of the clot, aspiration is either done through an aspiration catheter, or through a microcatheter for smaller arteries. Once the aspiration catheter is at the lesion, negative pressure is applied to remove the clot through the catheter. Alternatively, the clot can be removed by deploying a stent retriever through the microcatheter. Once the clot has integrated into the stent retriever, the clot is retrieved by retracting the stent retriever and microcatheter (or intermediate catheter) into the guide catheter.

In PCI, the physician uses a robotic system to gain lesion access by manipulating a coronary guidewire to deliver the therapy and restore normal blood flow. The access is enabled by seating a guide catheter in a coronary ostium. The distal tip of the guidewire is navigated past the lesion and, for complex anatomies, a microcatheter may be used to provide adequate support for the guidewire. The blood flow is restored by delivering and deploying a stent or balloon at the lesion. The lesion may need preparation prior to stenting, by either delivering a balloon for pre-dilation of the lesion, or by performing atherectomy using, for example, a laser or rotational atherectomy catheter and a balloon over the guidewire. Diagnostic imaging and physiological measurements may be performed to determine appropriate therapy by using imaging catheters or fractional flow reserve (FFR) measurements.

In PVI, the physician uses a robotic system to deliver the therapy and restore blood flow with techniques similar to NVI. The distal tip of the guidewire is navigated past the lesion and a microcatheter may be used to provide adequate support for the guidewire for complex anatomies. The blood flow is restored by delivering and deploying a stent or balloon to the lesion. As with PCI, lesion preparation and diagnostic imaging may be used as well.

When support at the distal end of a catheter or guidewire is needed, for example, to navigate tortuous or calcified vasculature, to reach distal anatomical locations, or to cross hard lesions, an over-the-wire (OTW) catheter or coaxial system is used. An OTW catheter has a lumen for the guidewire that extends the full length of the catheter. This provides a relatively stable system because the guidewire is supported along the whole length. This system, however, has some disadvantages, including higher friction, and longer overall length compared to rapid-exchange catheters (see below). Typically to remove or exchange an OTW catheter while maintaining the position of the indwelling guidewire, the exposed length (outside of the patient) of guidewire must be longer than the OTW catheter. A 300 cm long guidewire is typically sufficient for this purpose and is often referred to as an exchange length guidewire. Due to the length of the guidewire, two operators are needed to remove or exchange an OTW catheter. This becomes even more challenging if a triple coaxial, known in the art as a tri-axial system, is used (quadruple coaxial catheters have also been known to be used). However, due to its stability, an OTW system is often used in NVI and PVI procedures. On the other hand, PCI procedures often use rapid exchange (or monorail) catheters. The guidewire lumen in a rapid exchange catheter runs only through a distal section of the catheter, called the monorail or rapid exchange (RX) section. With a RX system, the operator manipulates the interventional devices parallel to each other (as opposed to with an OTW system, in which the devices are manipulated in a serial configuration), and the exposed length of guidewire only needs to be slightly longer than the RX section of the catheter. A rapid exchange length guidewire is typically 180-200 cm long. Given the shorter length guidewire and monorail, RX catheters can be exchanged by a single operator. However, RX catheters are often inadequate when more distal support is needed.

In a manual non-robotic procedure, a variety of wire-like EMDs, such as guidewires, stent retrievers, and coils, must be gripped by their shaft to linearly and/or rotationally manipulate the devices in the patient anatomy during a procedure. EMDs are typically gripped by the operator's fingers or with an off the shelf pin vice like device, commonly referred to as a torque device.

In a manual procedure a torquer is used by an operator to releasably pinch and unpinch a portion of an elongated medical device (EMD), such as a catheter guidewire, during a procedure. The torquer is used to releasably fix a portion of an EMD to allow a user to manipulate the EMD by rotating and/or translating the EMD.

### SUMMARY

An adaptor engaging a torquer, the adaptor including an adaptor body having a pathway therethrough. The adaptor body includes a receptacle portion defining a cavity having an opening at a first end of the adaptor body. The torquer is received within the cavity through the opening. The receptacle portion includes an engagement member securing the torquer to the adaptor body. The torquer is movable with the adaptor body along and about a longitudinal axis of the adaptor body.

In one embodiment a robotic drive system for a catheter procedure includes a device module including a drive member. An adaptor includes an adaptor body having a pathway extending therethrough. The adaptor body includes a receptacle portion defining a cavity having an opening at a first end of the adaptor body. A torquer is received within the cavity of the adaptor. The receptacle portion of the adaptor includes an engagement member securing the torquer to the adaptor body. A portion of the torquer is movable with the adaptor body along and about a longitudinal axis of the adaptor body. The adaptor body includes a driven member operatively connected to the drive member to rotate the adaptor body and torquer about a longitudinal axis of the adaptor.

In one embodiment a method for securing a torquer to a robotic drive system for a catheter procedure includes providing a device module including a drive member. Additionally, the method includes providing an adaptor including an adaptor body having a pathway extending therethrough and having a receptacle portion defining a cavity with an opening at a first end of the adaptor body. Additionally, the method includes attaching a torquer to the adaptor by inserting a nut of the torquer into the cavity of the adaptor through the opening at the first end of the adaptor body, while a portion of the torquer is located outside of the adaptor, wherein the nut is fixed to and movable with the adaptor body along and about a longitudinal axis of the adaptor body. Further the method includes securing an elongated medical device within the torquer and extending through the pathway and placing the elongated medical device, adaptor and torquer within the device module.

### BRIEF DESCRIPTION OF THE DRAWINGS

FIG. 1 is a schematic view of an exemplary catheter procedure system in accordance with an embodiment.

FIG. 2 is a schematic block diagram of an exemplary catheter procedure system in accordance with an embodiment.

FIG. 3 is an isometric view of an exemplary bedside system of a catheter procedure system in accordance with an embodiment.

FIG. 4 is an isometric view of an adaptor and torquer.

FIG. 5 is an exploded view of the adaptor and torquer in FIG. 4.

FIG. 6 is a longitudinal cross-section of the adaptor and torquer of FIG. 4 in a disengaged position.

FIG. 7 is a longitudinal cross-section of the adaptor and torquer of FIG. 4 in an engaged position.

FIG. 8 is a transverse cross-section of the adaptor and torquer taken along lines 8-8 of FIG. 6.

FIG. 9 is an isometric view of the device module and adaptor and torquer in an in-use position.

FIG. 10 is an is an exploded view of the device module and adaptor and torquer.

FIG. 11 is a plan view of the device module, adaptor and torquer in an in-use position.

FIG. 12 is an is an isometric view of an adaptor and a torquer.

### DETAILED DESCRIPTION OF THE EXAMPLE EMBODIMENTS

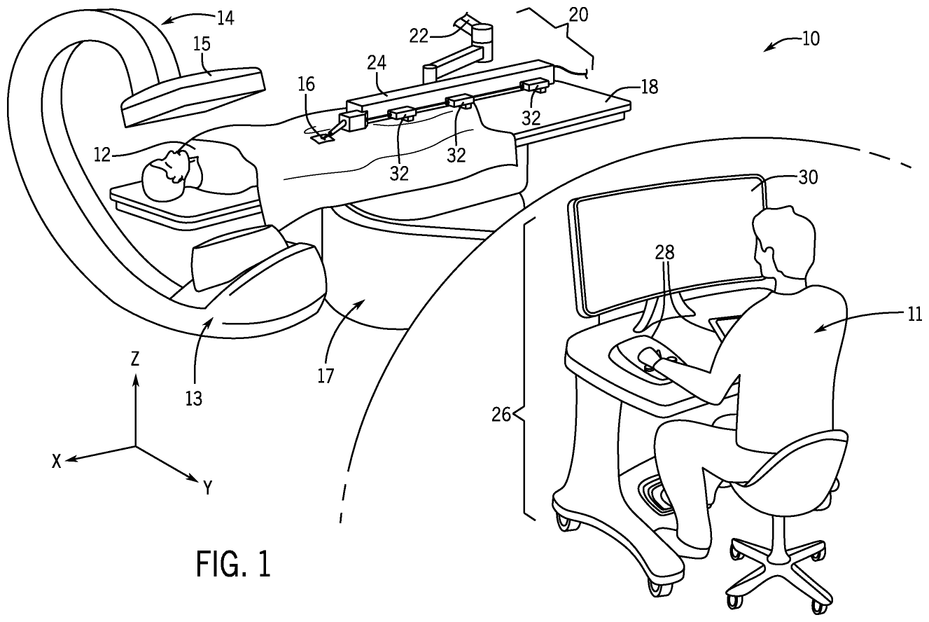

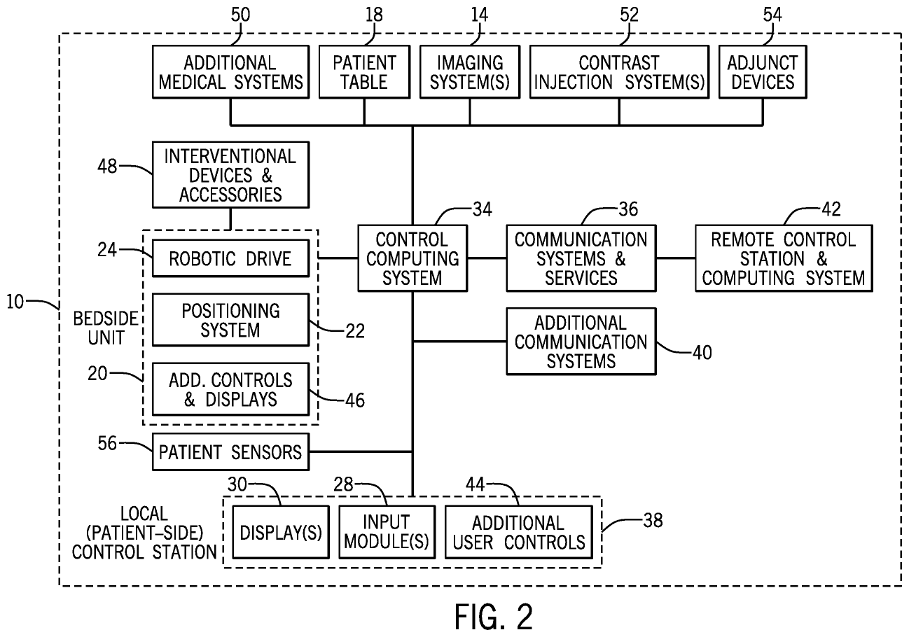

FIG. 1 is a perspective view of an exemplary catheter-based procedure system 10 in accordance with an embodiment. Catheter-based procedure system 10 may be used to perform catheter-based medical procedures, e.g., percutaneous intervention procedures such as a percutaneous coronary intervention (PCI) (e.g., to treat STEMI), a neurovascular interventional procedure (NVI) (e.g., to treat an emergent large vessel occlusion (ELVO)), peripheral vascular intervention procedures (PVI) (e.g., for critical limb ischemia (CLI), etc.). Catheter-based medical procedures may include diagnostic catheterization procedures during which one or more catheters or other elongated medical devices (EMDs) are used to aid in the diagnosis of a patient's disease. For example, during one embodiment of a catheter-based diagnostic procedure, a contrast media is injected onto one or more arteries through a catheter and an image of the patient's vasculature is taken. Catheter-based medical procedures may also include catheter-based therapeutic procedures (e.g., angioplasty, stent placement, treatment of peripheral vascular disease, clot removal, arterial venous malformation therapy, treatment of aneurysm, etc.) during which a catheter (or other EMD) is used to treat a disease. Therapeutic procedures may be enhanced by the inclusion of adjunct devices 54 (shown in FIG. 2) such as, for example, intravascular ultrasound (IVUS), optical coherence tomography (OCT), fractional flow reserve (FFR), etc. It should be noted, however, that one skilled in the art would recognize that certain specific percutaneous intervention devices or components (e.g., type of guidewire, type of catheter, etc.) may be selected based on the type of procedure that is to be performed. Catheter-based procedure system 10 can perform any number of catheter-based medical procedures with minor adjustments to accommodate the specific percutaneous intervention devices to be used in the procedure.

Catheter-based procedure system 10 includes, among other elements, a bedside unit 20 and a control station 26. Bedside unit 20 includes a robotic drive 24 and a positioning system 22 that are located adjacent to a patient 12. Patient 12 is supported on a patient table 18. The positioning system 22 is used to position and support the robotic drive 24. The positioning system 22 may be, for example, a robotic arm, an articulated arm, a holder, etc. The positioning system 22 may be attached at one end to, for example, a rail on the patient table 18, a base, or a cart. The other end of the positioning system 22 is attached to the robotic drive 24. The positioning system 22 may be moved out of the way (along with the robotic drive 24) to allow for the patient 12 to be placed on the patient table 18. Once the patient 12 is positioned on the patient table 18, the positioning system 22 may be used to situate or position the robotic drive 24 relative to the patient 12 for the procedure. In an embodiment, patient table 18 is operably supported by a pedestal 17, which is secured to the floor and/or earth. Patient table 18 is able to move with multiple degrees of freedom, for example, roll, pitch, and yaw, relative to the pedestal 17. Bedside unit 20 may also include controls and displays 46 (shown in FIG. 2). For example, controls and displays may be located on a housing of the robotic drive 24.

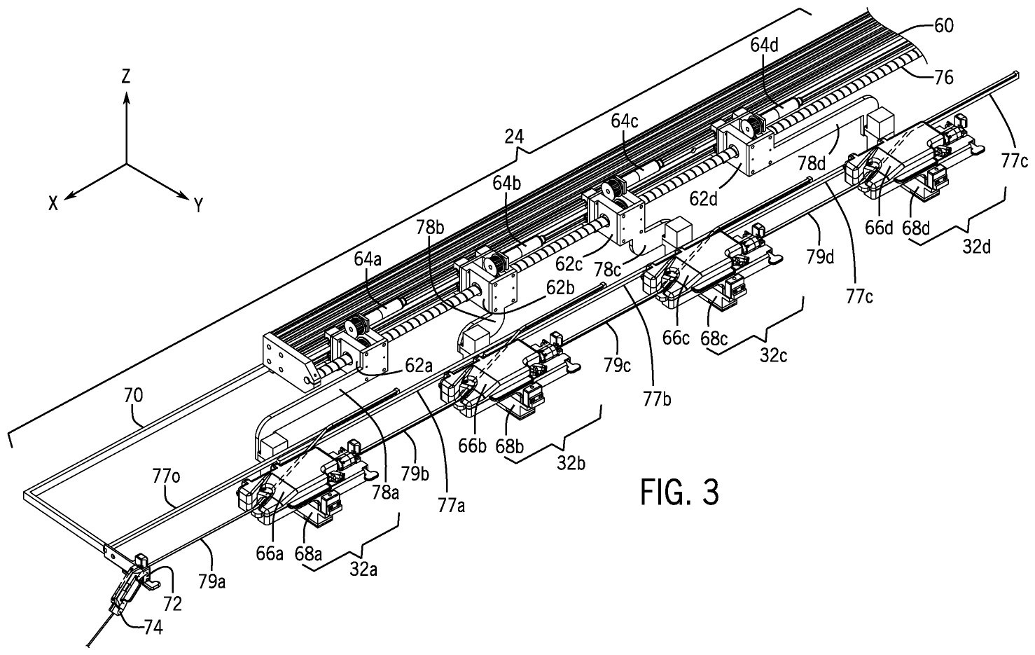

Generally, the robotic drive 24 may be equipped with the appropriate percutaneous interventional devices and accessories 48 (shown in FIG. 2) (e.g., guidewires, various types of catheters including balloon catheters, stent delivery systems, stent retrievers, embolization coils, liquid embolics, aspiration pumps, device to deliver contrast media, medicine, hemostasis valve adapters, syringes, stopcocks, inflation device, etc.) to allow the user or operator 11 to perform a catheter-based medical procedure via a robotic system by operating various controls such as the controls and inputs located at the control station 26. Bedside unit 20, and in particular robotic drive 24, may include any number and/or combination of components to provide bedside unit 20 with the functionality described herein. A user or operator 11 at control station 26 is referred to as the control station user or control station operator and referred to herein as user or operator. A user or operator at bedside unit 20 is referred to as bedside unit user or bedside unit operator. The robotic drive 24 includes a plurality of device modules 32a-d mounted to a rail or linear member 60 (shown in FIG. 3). The rail or linear member 60 guides and supports the device modules. Each of the device modules 32a-d may be used to drive an EMD such as a catheter or guidewire. For example, the robotic drive 24 may be used to automatically feed a guidewire into a diagnostic catheter and into a guide catheter in an artery of the patient 12. One or more devices, such as an EMD, enter the body (e.g., a vessel) of the patient 12 at an insertion point 16 via, for example, an introducer sheath.

Bedside unit 20 is in communication with control station 26, allowing signals generated by the user inputs of control station 26 to be transmitted wirelessly or via hardwire to bedside unit 20 to control various functions of bedside unit 20. As discussed below, control station 26 may include a control computing system 34 (shown in FIG. 2) or be coupled to the bedside unit 20 through a control computing system 34. Bedside unit 20 may also provide feedback signals (e.g., loads, speeds, operating conditions, warning signals, error codes, etc.) to control station 26, control computing system 34 (shown in FIG. 2), or both. Communication between the control computing system 34 and various components of the catheter-based procedure system 10 may be provided via a communication link that may be a wireless connection, cable connections, or any other means capable of allowing communication to occur between components. Control station 26 or other similar control system may be located either at a local site (e.g., local control station 38 shown in FIG. 2) or at a remote site (e.g., remote control station and computer system 42 shown in FIG. 2). Catheter procedure system 10 may be operated by a control station at the local site, a control station at a remote site, or both the local control station and the remote control station at the same time. At a local site, user or operator 11 and control station 26 are located in the same room or an adjacent room to the patient 12 and bedside unit 20. As used herein, a local site is the location of the bedside unit 20 and a patient 12 or subject (e.g., animal or cadaver) and the remote site is the location of a user or operator 11 and a control station 26 used to control the bedside unit 20 remotely. A control station 26 (and a control computing system) at a remote site and the bedside unit 20 and/or a control computing system at a local site may be in communication using communication systems and services 36 (shown in FIG. 2), for example, through the Internet. In an embodiment, the remote site and the local (patient) site are away from one another, for example, in different rooms in the same building, different buildings in the same city, different cities, or other different locations where the remote site does not have physical access to the bedside unit 20 and/or patient 12 at the local site.

Control station 26 generally includes one or more input modules 28 configured to receive user inputs to operate various components or systems of catheter-based procedure system 10. In the embodiment shown, control station 26 allows the user or operator 11 to control bedside unit 20 to perform a catheter-based medical procedure. For example, input modules 28 may be configured to cause bedside unit 20 to perform various tasks using percutaneous intervention devices (e.g., EMDs) interfaced with the robotic drive 24 (e.g., to advance, retract, or rotate a guidewire, advance, retract or rotate a catheter, inflate or deflate a balloon located on a catheter, position and/or deploy a stent, position and/or deploy a stent retriever, position and/or deploy an embolization coil, inject contrast media into a catheter, inject liquid embolics into a catheter, inject medicine or saline into a catheter, aspirate on a catheter, or to perform any other function that may be performed as part of a catheter-based medical procedure). Robotic drive 24 includes various drive mechanisms to cause movement (e.g., axial and rotational movement) of the components of the bedside unit 20 including the percutaneous intervention devices.

In one embodiment, input modules 28 may include one or more touch screens, joysticks, scroll wheels, and/or buttons. In addition to input modules 28, the control station 26 may use additional user controls 44 (shown in FIG. 2) such as foot switches and microphones for voice commands, etc. Input modules 28 may be configured to advance, retract, or rotate various components and percutaneous intervention devices such as, for example, a guidewire, and one or more catheters or microcatheters. Buttons may include, for example, an emergency stop button, a multiplier button, device selection buttons and automated move buttons. When an emergency stop button is pushed, the power (e.g., electrical power) is shut off or removed to bedside unit 20. When in a speed control mode, a multiplier button acts to increase or decrease the speed at which the associated component is moved in response to a manipulation of input modules 28. When in a position control mode, a multiplier button changes the mapping between input distance and the output commanded distance. Device selection buttons allow the user or operator 11 to select which of the percutaneous intervention devices loaded into the robotic drive 24 are controlled by input modules 28. Automated move buttons are used to enable algorithmic movements that the catheter-based procedure system 10 may perform on a percutaneous intervention device without direct command from the user or operator 11. In one embodiment, input modules 28 may include one or more controls or icons (not shown) displayed on a touch screen (that may or may not be part of a display 30), that, when activated, causes operation of a component of the catheter-based procedure system 10. Input modules 28 may also include a balloon or stent control that is configured to inflate or deflate a balloon and/or deploy a stent. Each of the input modules 28 may include one or more buttons, scroll wheels, joysticks, touch screen, etc. that may be used to control the particular component or components to which the control is dedicated. In addition, one or more touch screens may display one or more icons (not shown) related to various portions of input modules 28 or to various components of catheter-based procedure system 10.

Control station 26 may include a display 30. In other embodiments, the control station 26 may include two or more displays 30. Display 30 may be configured to display information or patient specific data to the user or operator 11 located at control station 26. For example, display 30 may be configured to display image data (e.g., X-ray images, MRI images, CT images, ultrasound images, etc.), hemodynamic data (e.g., blood pressure, heart rate, etc.), patient record information (e.g., medical history, age, weight, etc.), lesion or treatment assessment data (e.g., IVUS, OCT, FFR, etc.). In addition, display 30 may be configured to display procedure specific information (e.g., procedural checklist, recommendations, duration of procedure, catheter or guidewire position, volume of medicine or contrast agent delivered, etc.). Further, display 30 may be configured to display information to provide the functionalities associated with control computing system 34 (shown in FIG. 2). Display 30 may include touch screen capabilities to provide some of the user input capabilities of the system.

Catheter-based procedure system 10 also includes an imaging system 14. Imaging system 14 may be any medical imaging system that may be used in conjunction with a catheter based medical procedure (e.g., non-digital X-ray, digital X-ray, CT, MRI, ultrasound, etc.). In an exemplary embodiment, imaging system 14 is a digital X-ray imaging device that is in communication with control station 26. In one embodiment, imaging system 14 may include a C-arm (shown in FIG. 1) that allows imaging system 14 to partially or completely rotate around patient 12 in order to obtain images at different angular positions relative to patient 12 (e.g., sagittal views, caudal views, anterior-posterior views, etc.). In one embodiment imaging system 14 is a fluoroscopy system including a C-arm having an X-ray source 13 and a detector 15, also known as an image intensifier.

Imaging system 14 may be configured to take X-ray images of the appropriate area of patient 12 during a procedure. For example, imaging system 14 may be configured to take one or more X-ray images of the head to diagnose a neurovascular condition. Imaging system 14 may also be configured to take one or more X-ray images (e.g., real time images) during a catheter-based medical procedure to assist the user or operator 11 of control station 26 to properly position a guidewire, guide catheter, microcatheter, stent retriever, coil, stent, balloon, etc. during the procedure. The image or images may be displayed on display 30. For example, images may be displayed on display 30 to allow the user or operator 11 to accurately move a guide catheter or guidewire into the proper position.

In order to clarify directions, a rectangular coordinate system is introduced with X, Y, and Z axes. The positive X axis is oriented in a longitudinal (axial) distal direction, that is, in the direction from the proximal end to the distal end, stated another way from the proximal to distal direction. The Y and Z axes are in a transverse plane to the X axis, with the positive Z axis oriented up, that is, in the direction opposite of gravity, and the Y axis is automatically determined by right-hand rule.

FIG. 2 is a block diagram of catheter-based procedure system 10 in accordance with an exemplary embodiment. Catheter-procedure system 10 may include a control computing system 34. Control computing system 34 may physically be, for example, part of control station 26 (shown in FIG. 1). Control computing system 34 may generally be an electronic control unit suitable to provide catheter-based procedure system 10 with the various functionalities described herein. For example, control computing system 34 may be an embedded system, a dedicated circuit, a general-purpose system programmed with the functionality described herein, etc. Control computing system 34 is in communication with bedside unit 20, communications systems and services 36 (e.g., Internet, firewalls, cloud services, session managers, a hospital network, etc.), a local control station 38, additional communications systems 40 (e.g., a telepresence system), a remote control station and computing system 42, and patient sensors 56 (e.g., electrocardiogram (ECG) devices, electroencephalogram (EEG) devices, blood pressure monitors, temperature monitors, heart rate monitors, respiratory monitors, etc.). The control computing system is also in communication with imaging system 14, patient table 18, additional medical systems 50, contrast injection systems 52 and adjunct devices 54 (e.g., IVUS, OCT, FFR, etc.). The bedside unit 20 includes a robotic drive 24, a positioning system 22 and may include additional controls and displays 46. As mentioned above, the additional controls and displays may be located on a housing of the robotic drive 24. Interventional devices and accessories 48 (e.g., guidewires, catheters, etc.) interface to the bedside system. In an embodiment, interventional devices and accessories 48 may include specialized devices (e.g., IVUS catheter, OCT catheter, FFR wire, diagnostic catheter for contrast, etc.) which interface to their respective adjunct devices 54, namely, an IVUS system, an OCT system, and FFR system, etc.

In various embodiments, control computing system 34 is configured to generate control signals based on the user's interaction with input modules 28 (e.g., of a control station 26 (shown in FIG. 1) such as a local control station 38 or a remote control station 42) and/or based on information accessible to control computing system 34 such that a medical procedure may be performed using catheter-based procedure system 10. The local control station 38 includes one or more displays 30, one or more input modules 28, and additional user controls 44. The remote control station and computing system 42 may include similar components to the local control station 38. The remote 42 and local 38 control stations can be different and tailored based on their required functionalities. The additional user controls 44 may include, for example, one or more foot input controls. The foot input control may be configured to allow the user to select functions of the imaging system 14 such as turning on and off the X-ray and scrolling through different stored images. In another embodiment, a foot input device may be configured to allow the user to select which devices are mapped to scroll wheels included in input modules 28. Additional communication systems 40 (e.g., audio conference, video conference, telepresence, etc.) may be employed to help the operator interact with the patient, medical staff (e.g., angio-suite staff), and/or equipment in the vicinity of the bedside.

Catheter-based procedure system 10 may be connected or configured to include any other systems and/or devices not explicitly shown. For example, catheter-based procedure system 10 may include image processing engines, data storage and archive systems, automatic balloon and/or stent inflation systems, medicine injection systems, medicine tracking and/or logging systems, user logs, encryption systems, systems to restrict access or use of catheter-based procedure system 10, etc.

As mentioned, control computing system 34 is in communication with bedside unit 20 which includes a robotic drive 24, a positioning system 22 and may include additional controls and displays 46 and may provide control signals to the bedside unit 20 to control the operation of the motors and drive mechanisms used to drive the percutaneous intervention devices (e.g., guidewire, catheter, etc.). The various drive mechanisms may be provided as part of a robotic drive 24. FIG. 3 is a perspective view of a robotic drive for a catheter-based procedure system 10 in accordance with an embodiment. In FIG. 3, a robotic drive 24 includes multiple device modules 32a-d coupled to a linear member 60. Each device module 32a-d is coupled to the linear member 60 via a stage 62a-d moveably mounted to the linear member 60. A device module 32a-d may be connected to a stage 62a-d using a connector such as an offset bracket 78a-d. In another embodiment, the device module 32a-d is directly mounted to the stage 62a-d. Each stage 62a-d may be independently actuated to move linearly along the linear member 60. Accordingly, each stage 62a-d (and the corresponding device module 32a-d coupled to the stage 62a-d) may independently move relative to each other and the linear member 60. A drive mechanism is used to actuate each stage 62a-d. In the embodiment shown in FIG. 3, the drive mechanism includes independent stage translation motors 64a-d coupled to each stage 62a-d and a stage drive mechanism 76, for example, a lead screw via a rotating nut, a rack via a pinion, a belt via a pinion or pulley, a chain via a sprocket, or the stage translation motors 64a-d may be linear motors themselves. In some embodiments, the stage drive mechanism 76 may be a combination of these mechanisms, for example, each stage 62a-d could employ a different type of stage drive mechanism. In an embodiment where the stage drive mechanism is a lead screw and rotating nut, the lead screw may be rotated and each stage 62a-d may engage and disengage from the lead screw to move, e.g., to advance or retract. In the embodiment shown in FIG. 3, the stages 62a-d and device modules 32a-d are in a serial drive configuration.

Each device module 32a-d includes a drive module 68a-d and a cassette 66a-d mounted on and coupled to the drive module 68a-d. In the embodiment shown in FIG. 3, each cassette 66a-d is mounted to the drive module 68a-d in a vertical orientation. In other embodiments, each cassette 66a-d may be mounted to the drive module 68a-d in other mounting orientations. Each cassette 66a-d is configured to interface with and support a proximal portion of an EMD (not shown). In addition, each cassette 66a-d may include elements to provide one or more degrees of freedom in addition to the linear motion provided by the actuation of the corresponding stage 62a-d to move linearly along the linear member 60. For example, the cassette 66a-d may include elements that may be used to rotate the EMD when the cassette is coupled to the drive module 68a-d. Each drive module 68a-d includes at least one coupler to provide a drive interface to the mechanisms in each cassette 66a-d to provide the additional degree of freedom. Each cassette 66a-d also includes a channel in which a device support 79a-d is positioned, and each device support 79a-d is used to prevent an EMD from buckling. A support arm 77a, 77b, and 77c is attached to each device module 32a, 32b, and 32c, respectively, to provide a fixed point for support of a proximal end of the device supports 79b, 79c, and 79d, respectively. The robotic drive 24 may also include a device support connection 72 connected to a device support 79, a distal support arm 70 and a support arm 77o. Support arm 77o is used to provide a fixed point for support of the proximal end of the distal most device support 79a housed in the distal most device module 32a. In addition, an introducer interface support (redirector) 74 may be connected to the device support connection 72 and an EMD (e.g., an introducer sheath). The configuration of robotic drive 24 has the benefit of reducing volume and weight of the drive robotic drive 24 by using actuators on a single linear member.

To prevent contaminating the patient with pathogens, healthcare staff use aseptic technique in a room housing the bedside unit 20 and the patient 12 or subject (shown in FIG. 1). A room housing the bedside unit 20 and patient 12 may be, for example, a cath lab or an angio suite. Aseptic technique consists of using sterile barriers, sterile equipment, proper patient preparation, environmental controls and contact guidelines. Accordingly, all EMDs and interventional accessories are sterilized and can only be in contact with either sterile barriers or sterile equipment. In an embodiment, a sterile drape (not shown) is placed over the non-sterile robotic drive 24. Each cassette 66a-d is sterilized and acts as a sterile interface between the draped robotic drive 24 and at least one EMD. Each cassette 66a-d can be designed to be sterile for single use or to be re-sterilized in whole or part so that the cassette 66a-d or its components can be used in multiple procedures.

The term buckling refers to the tendency of a flexible EMD when under axial compression to undesirably bend away from the longitudinal axis or intended path along which it is being advanced. In one embodiment axial compression occurs in response to resistance from being navigated in the vasculature. The distance an EMD may be driven along its longitudinal axis without support before the EMD buckles is referred to herein as the device buckling distance. The device buckling distance is a function of the device's stiffness, geometry (including but not limited to diameter), and force being applied to the EMD. Buckling may cause the EMD to form an arcuate portion different than the intended path. Kinking is a case of buckling in which deformation of the EMD is non-elastic resulting in a permanent set.

The term device module refers to the combination of a drive module and a cassette.

The term longitudinal axis of a member (for example, an EMD or other element in the catheter-based procedure system) is the line or axis along the length of the member that passes through the center of the transverse cross section of the member in the direction from a proximal portion of the member to a distal portion of the member. For example, the longitudinal axis of a guidewire is the central axis in the direction from a proximal portion of the guidewire toward a distal portion of the guidewire even though the guidewire may be non-linear in the relevant portion.

The terms user or operator refer to a user or operator at a control station. The terms also refer to as a control station user or control station operator.

The term cassette generally refers to the part (non-capital, consumable or sterilizable unit) of the robotic drive system that normally is the sterile interface between a drive module and at least one EMD (directly) or through a device adapter (indirectly).

The term axial movement of a member refers to translation of the member along the longitudinal axis of the member and the term axial insertion refers to inserting a first member into a second member along the longitudinal axis of the second member. The term torquer refers to a device such as a collet that can releasably fix a portion of an EMD. The term fixed here means no intentional relative movement of the collet and EMD during operation. The terms distal and proximal define relative locations of two different features. With respect to a robotic drive the terms distal and proximal are defined by the position of the robotic drive in its intended use relative to a patient.

When used to define a relative position, the distal feature is the feature of the robotic drive that is closer to the patient than a proximal feature when the robotic drive is in its intended in-use position. Within a patient, any vasculature landmark further away along the path from the access point is considered more distal than a landmark closer to the access point, where the access point is the point at which the EMD enters the patient. Similarly, the proximal feature is the feature that is farther from the patient than the distal feature when the robotic drive in its intended in-use position. When used to define direction, the distal direction refers to a path on which something is moving or is aimed to move or along which something is pointing or facing from a proximal feature toward a distal feature and/or patient when the robotic drive is in its intended in-use position. The proximal direction is the opposite direction of the distal direction.

The term elongated medical device (EMD) refers to, but is not limited to, catheters (e.g., guide catheters, microcatheters, balloon/stent catheters), wire-based devices (e.g., guidewires, embolization coils, stent retrievers, etc.), and medical devices comprising any combination of these.

The term fixed means no intentional relative movement of a first member with respect to a second member during operation.

The term rotational movement of a member refers to the change in angular orientation of the member about the local longitudinal axis of the member.

The term pinch refers to releasably fixing an EMD to a member such that the EMD and member move together when the member moves. Rotational movement of the member will result in rotational movement of the EMD in the pinched condition. The term unpinch refers to releasing the EMD from a member such that the EMD and member move independently when the member moves. In an unpinched condition the EMD can be moved/rotated relative to the member.

The term collet refers to a device that can releasably fix a portion of an EMD. The term fixed here means no intentional relative movement of the collet and EMD during operation.

The term torquer refers to a device that releasably pinches and unpinches a portion of an EMD, such as a guidewire. The term torquer is a generally accepted term used by medical professionals in catheter procedures to indicate a device used to rotate an EMD and/or translate an EMD. A torquer is also known generally as a collet or pin-vice. Torquers described herein are used ex vivo to pinch a portion of an EMD outside of the patient's body.

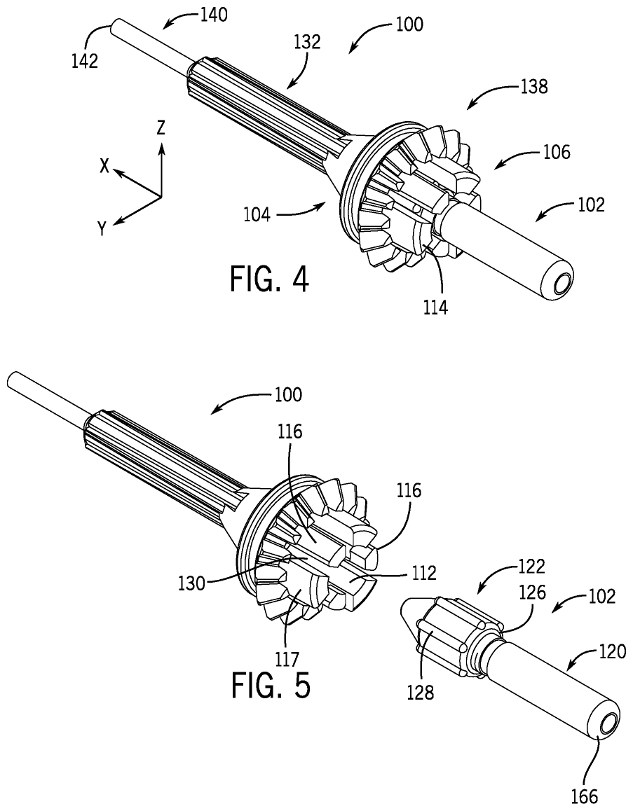

Referring to FIGS. 4-6 an adaptor 100 engages a torquer 102. Adaptor 100 includes an adaptor body 104 having a pathway 106 therethrough. Body 104 includes a receptacle portion 108 defining a cavity 110 having an opening 112 at a first end 114 of the adaptor body 104. Torquer 102 is received within cavity 110 through opening 112. Receptacle portion 108 includes an engagement mechanism member 116 securing torquer 102 to adaptor body 104. Torquer 102 is movable with adaptor body 104 along and about a longitudinal axis 118 of adaptor body 104. In one embodiment engagement mechanism 116 includes a single engagement member and in one embodiment there is more than one engagement member. The receptacle portion includes anti-rotation features such that at least a portion of the torquer does not rotate independently of the adaptor. The anti-rotational features are described below. Referring to FIG. 8 in one embodiment there are three engagement members 116a, 116b and 116c. In one embodiment receptable portion may include other mechanisms to engage the torquer. In one embodiment a portion of the torquer may be affixed to the adaptor by press fitting, ultrasound, heat, vibration welding, bonding, or mechanisms known in the art.

Torquer 102 includes a torquer body 120 and a nut 122 rotatable relative to torquer body 120. Rotation of nut 122 engages and/or disengages an EMD such that the EMD is fixed relative to the torquer 102. In an engaged position axial along and/or rotational movement about a longitudinal axis of the torquer will result in corresponding axial and/or rotational movement of the EMD. In one embodiment torquer 102 is an off the shelf device such as the GLIDEWIRE® TORQUE™ Device. In one embodiment torquer 102 is a torque device sold by Merit Medical under the name MERIT® Torque Device or Merit MAP500. Other off the shelf torquer devices may also be used. In one embodiment the off the shelf torquer can be used manually or within a robotic system. In one embodiment torquer 102 maybe a specialty torquer with a nut designed to be connected to the adaptor such that the nut is translated along and rotated about the longitudinal axis of the adaptor. In one embodiment the nut of the torquer is integrally formed with an adaptor having a driven member, a grip portion. In one embodiment the adaptor with an integral nut also includes an extension member portion as described herein. The adaptor and integral nut will be rotatably connected to the torquer body to move at least one jaw from a disengaged position in which an EMD is not fixed to the torquer to an engaged position in which the EMD is fixed to the torquer.

In one embodiment nut 122 is snap fit into cavity 110 of receptacle portion 108. The term snap fit is an assembly method used to attach flexible parts, usually plastic, to form the final product by pushing the parts' interlocking components together. There are a number of variations in snap-fits, including cantilever, torsional and annular. Snap-fits, as integral attachment features, are an alternative to assembly using nails or screws, and have the advantages of speed and no loose parts. Engagement member 116 is a cantilever design snap fit defining a lever having a tab 124 that engages a proximal portion 126 of nut 122 to fixedly engage torquer 102 with adaptor 100. Proximal portion 126 may be a shelf feature having a profile substantially perpendicular to longitudinal axis 118. Proximal portion 126 may also be a recess that receives tab 124. As nut 122 is moved into cavity 110 in a direction along longitudinal axis 118 engagement member 116 moves radially away from longitudinal axis 118 until tab 124 is free to engage proximal portion 126 thereby allowing engagement member 116 to move radially toward longitudinal axis 118. In one embodiment nut 122 includes at least one member 128 received within a slot 130 defined by the receptacle portion to prevent independent rotation of nut 122 relative to adaptor 100 about longitudinal axis 118. Stated another way engagement member 116 includes at least one tab 124 having a free end that snap fits over nut 122. In one embodiment engagement member 116 is integrally formed with adaptor body 104. Slot 130 is defined as the space between one engagement member 116 and a second member 117. Referring to FIG. 5 in one embodiment engagement member includes three separate members 116 and three spaced members 117 interspaced therebetween such that each spaced member 117 is between adjacent members 116.

In one embodiment torquer nut 122 is removable from the adaptor body without damaging a portion of engagement member 116 and/or a portion of nut 122. In one embodiment torquer nut 122 is not removable from adaptor body without damaging a portion of engagement member 116 and/or a portion of nut 122.

Referring to FIG. 7 adaptor body 104 includes a grip portion 132 extending along longitudinal axis 118 of adaptor body 104 in a direction away from receptacle portion 108 and the opening 112 of cavity 110. In one embodiment adaptor body 104 includes a beveled transition between grip portion 132 and opening 112 such that the outer diameter of the grip portion is less than the outer diameter of the receptacle portion.

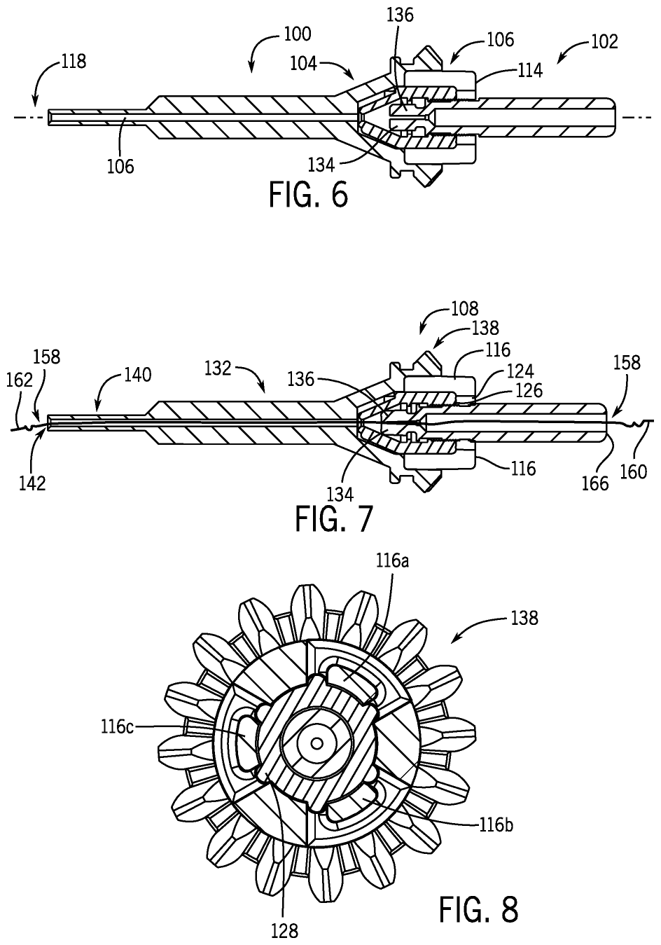

Referring to FIGS. 6 and 7, torquer 102 can be moved to the engaged position to fixedly engage an EMD after torquer has been snap fit into adaptor body 104. Nut 122 is rotated relative to the torquer body 120 by rotating grip portion 132 of adapter 100 relative to the torquer body 120 thereby engaging an EMD between a first jaw 134 and a second jaw 136 of torquer 102.

In one embodiment adaptor 100 includes a driven member 138. Driven member 138 in one embodiment is a beveled gear that engages with a drive gear in a robotic system such as robotic system 10. Driven member 138 in one embodiment is integrally formed with the adaptor body 104. Driven member 138 in one embodiment is intermediate the grip portion 132 and opening 112 of receptacle portion 108.

In one embodiment adaptor 100 includes an extension member portion 140 extending from grip portion 132 in a direction away from opening 112 of cavity 110. Extension member provides anti-buckling support for an EMD between torquer 102 and a support track. Extension member portion 140 is part of adaptor body 104 having a free end 142 defining a second end of the adaptor 100 opposite the first end 114. In one embodiment outer diameter 144 of grip portion 132 is greater than the outer diameter 146 of extension member portion 140.

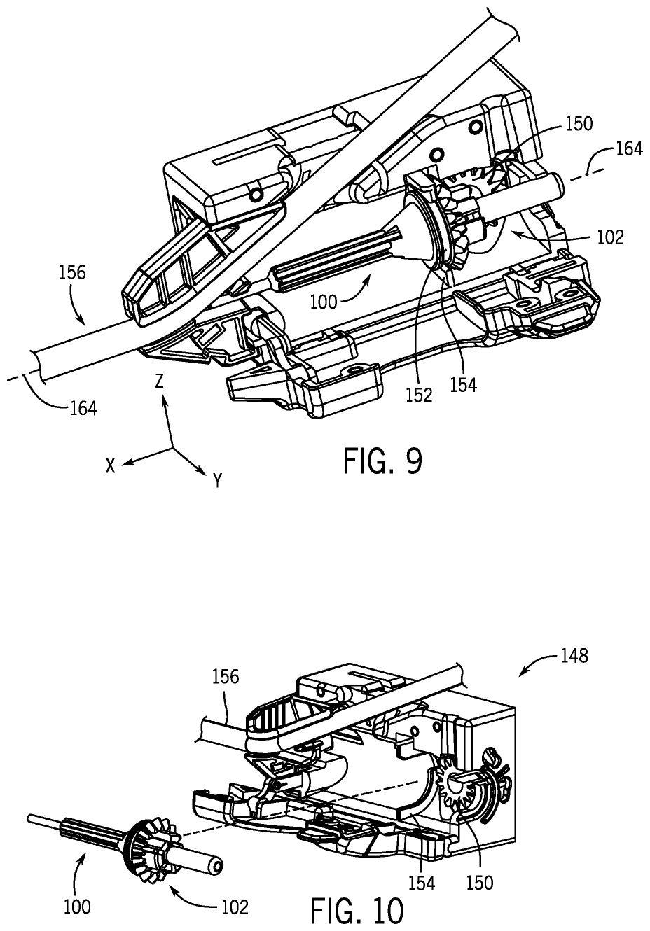

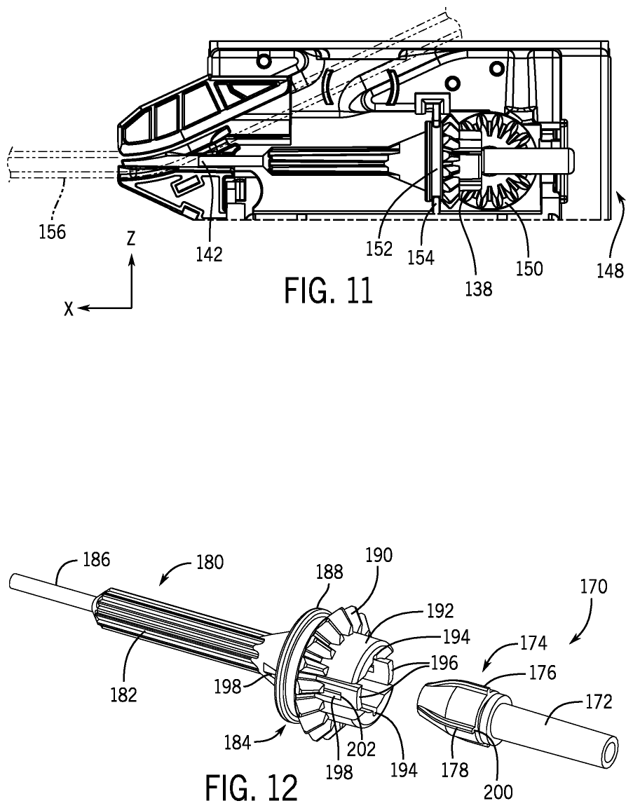

Referring to FIGS. 9, 10 and 11 adaptor 100 and torquer 102 are positioned within a device module 148 having a drive member 150 of catheter based producer system. Adaptor 100 and torquer 102 are in the engaged position with nut 122 of torquer 102 within cavity 110. Device module 148 is an embodiment of device module 32 described in connection with FIGS. 1-3 and can be used with catheter procedure system 10. As described above, adaptor 100 includes an adaptor body 104 having a pathway 106 therethrough and a receptacle portion 108 defining a cavity 110 having an opening 112 at a first end 114 of the adaptor body 104. First end 114 of adaptor body is the proximal end of the adaptor body 104. Receptacle portion 108 includes an engagement member 116 securing the torquer 102 to the adaptor body 104. Once torquer 102 is in the engaged position, torquer 102 is movable with adaptor body 104 along and about a longitudinal axis 118 of adaptor body 104. In one embodiment the longitudinal axis 118 of adaptor body 104 when the adaptor is located in an in-use position in the device module 148 is co-linear with the longitudinal axis of device module 148.

Adaptor 100 and torquer 102 have the same features as described above for use in the device module 148. In one embodiment adaptor body 104 includes an outer bearing portion 152 that is rotatably received with a device module bearing surface 154. Device module bearing surface provides rotational and thrust support of the adaptor 100 and torquer 102 such that adaptor 100 and torquer 102 can rotate about the longitudinal axis of the device module while the device module itself does not rotate. In one embodiment device module bearing surface provides axial support of adaptor 100 and torquer 102 such that adaptor 100 and torquer 102 remain fixed along the longitudinal axis of the device module. When the adaptor 100 and torquer 102 are in the in-use position in the device module a proximal end of torquer body 120 is positioned proximal to the nut 122 and drive member 150. In the in-use position adaptor body proximal end of adaptor 100 is positioned distal to the proximal end of the proximal end of torquer body 120.

With the adaptor 100 and torquer 102 in the in-use position within the device module jaws within the torquer can move between an engaged position and disengaged position operatively pinching an EMD thereto by rotating the adaptor body and nut together relative to the torquer body. Since the nut is rotationally fixed to adaptor body rotation of the adaptor body will rotate the nut therewith. By rotating the grip portion of the adaptor body relative to the torquer body in a first direction jaws of the torquer will move from a disengaged position in which the EMD is not fixed to the jaws to an engaged position in which the EMD is fixed to the jaws. The term fixed means that movement of the jaws along or about the longitudinal axis of the device module will result in corresponding movement of the EMD along and/or about the longitudinal axis of the device module.

The distal end of extension member portion that extends from the grip portion in a distal direction away from the opening of the cavity defines a free end of the adaptor. The free end of the extension member portion is positioned closely adjacent to a track when the adaptor is in the in-use position within the device module. In one embodiment the distal free end of the extension member portion is closely adjacent to the device support or flexible track along the longitudinal axis of the device module such that the EMD does not buckle between the distal end of the extension member and the track when the EMD is being translated and/or rotated. In one embodiment the distance between the distal free end of the extension member portion and the device support is less than one inch and in one embodiment less than 0.5 inches (12.7 mm). In one embodiment the distal fee end of the extension member portion is located within the lumen defined by the device support or track. In one embodiment track is formed from a flexible member that moves from a position co-linear to the longitudinal axis of the device module to a position off set from the longitudinal axis of the device module as the device module moves relative to the track. In one embodiment the portion of the EMD within torquer 102 adaptor 100 and track 156 is in a straight line. In one embodiment the portion of the EMD in a straight line also includes the portion of the EMD extending through the track to closely adjacent the patient.

In use an EMD such as a guidewire extends through the torquer 102 and pathway of the adaptor 100. The EMD is fixed to the torquer 102 such that movement of the torquer along or about the longitudinal axis 164 of the device module results in corresponding movement of the EMD along or about the longitudinal axis 164 of the device module. In one embodiment, rotation of the torquer 102 is robotically controlled by a signal to rotate the drive member which engages the driven member thereby rotating the adaptor 100 and torquer 102 and the EMD fixed to the torquer 102. In one embodiment movement of the device module along the longitudinal axis of the device module in the distal and/or proximal direction results in movement along the longitudinal axis of the EMD in the corresponding distal and/or proximal direction.

The distal end of extension member portion that extends from the grip portion in a distal direction away from the opening of the cavity defines a free end of the adaptor. The free end of the extension member portion is positioned closely adjacent to a device support flexible track when the adaptor is in the in-use position within the device module.

In one embodiment a method for securing a torquer to a robotic drive system such as catheter based procedure system 10 includes providing a device module 32 such as device module 148 that includes a drive member 150. An adaptor 100 including an adaptor body 104 having a pathway 106 extending therethrough and a receptacle portion defining a cavity having an opening at a first end of the adaptor body. Torquer 102 is secured to the adaptor 100 by inserting a nut 122 of torquer 102 into cavity 110 of adaptor 100 through opening 112 at the first end 114 or proximal end of adaptor body 104. A portion of torquer 102 is located outside of the adaptor body 104. Nut 122 is fixed to and movable with adaptor body 104 along and about a longitudinal axis of the adaptor body. In one embodiment nut is snap fit into cavity 110 by engagement member 116. In one embodiment engagement member 116 is integrally formed with adaptor body such that a free end of engagement member 116 can extend in a direction radially away from the longitudinal axis 118 along an outer surface of the adaptor body and then then move radially toward the longitudinal axis 118 to engage a portion of torquer body 120 such as a shoulder portion of nut 122 or other recess or detent.

A proximal end 160 of an elongated medical device such as a guidewire is inserted into the distal opening of torquer body 120 and pushed through and out of the proximal opening of nut 122. A portion of the guidewire intermediate a proximal end of the guidewire and distal end of the guidewire is fixed to the torquer 102 as is understood by one of ordinary skill in the art including but not limited to movement of a nut relative to the torquer housing to move at least one jaw toward the elongated medical device to fix it to the torquer. Once the guide wire is thread through the torquer, the proximal end of the guide wire is moved through a proximal end of adaptor body and through the distal end opening of the adaptor housing extending such that a portion of the guidewire is positioned with the adaptor housing.

In one embodiment after the torquer is placed within the adaptor and the elongated medical device extends through both the torquer and adaptor, the adaptor torquer and elongated medical device is positioned within the device module. In one embodiment it is contemplated that the elongated medical device is positioned through the torquer and adaptor after the torquer and adaptor are positioned within the device module. In one embodiment the EMD is front or proximally loaded by inserting a distal end of the EMD first into an opening at the proximal end of the torquer and then through the torquer and through the adaptor pathway until the distal end of the EMD extends through the opening of the distal end of the adaptor. In one embodiment the EMD is backloaded by inserting the proximal end of the EMD first through the distal opening of the adaptor and pushing the proximal end of the EMD through the pathway and through the torquer until the proximal end of the EMD exits through the proximal opening of the torquer. It is also contemplated in one embodiment that the adaptor and torquer has a slit extending the entire length of the adaptor and torquer from an outer surface of the adaptor and torquer to the pathway of the adaptor and lumen of the torquer. An EMD in this embodiment can be inserted into the adaptor and lumen by passing a portion of the EMD intermediate a proximal end and distal end of the EMD through the slits into the pathway and lumen.

The driven member of the adaptor is engaged with the drive member of the device module and a user controls the rotational movement of the elongated medical device by operatively rotating the drive member which rotates the driven member, adaptor, torquer and elongated medical device together. In one embodiment nut 122 is integrally formed with adaptor 100.

Referring to FIG. 12, another torquer 170 is connected to an adaptor 180. In one embodiment torquer 170 is an off the shelf commercially available torquer used in manual procedures sold by Merit described above and also known as the Merit MAP500 or the MERIT Torque Device. Torquer 170 includes a torquer body 172 and a torquer nut 174 threadedly attached to torquer body 172 to move a pair of jaws sufficiently toward and away from a longitudinal axis of the torquer body 172 to engage and disengage an elongated medical device such as a guidewire. In an engaged position the EMD is fixed to the torquer such that the EMD and moves axially and rotationally about the longitudinal axis of the torquer longitudinal axis with the torquer. Torquer nut 174 includes a first pair of ribs 176 and a second pair of ribs 178 extending along an outer surface of the torquer nut from a distal end toward a proximal end of the torquer nut 174. Adaptor 180 is similar to adaptor 100 with the receptacle portion and engagement mechanism being different. Adaptor includes an adaptor body having a grip portion 182, and extension portion 186 extending distally from grip portion 182, a receptacle portion 184, a bearing portion 188 and a driven member 190. Receptacle portion 184 includes a cavity receiving torquer nut 174 and an opening on the proximal end of the adaptor 180. A lumen in fluid communication with the cavity extends through the adaptor grip portion 182 and the adaptor extension portion 186. In the installed position the longitudinal axis of torquer 170 is co-axial with a longitudinal axis of the adaptor 180 and co-axial with the cavity and the lumen. As described above an EMD such as a guidewire extends through the cavity and lumen. Receptacle portion includes a first pair of members 192 having a free end proximate the opening and a second pair of engagement members 196 having a free end proximate the opening. Each of the first pair of members 192 include a groove 194 through which a respective first rib 176 is received when torquer is connected to adaptor 180. Each of second pair of members 196 include a slot 198 having a proximal end that is spaced from the proximal free end of member 196. Second pair of members 196 form a cantilever such that the free proximal end of members 196 that move radially outward away from the longitudinal axis of the adaptor as the torquer is moved through the opening into the cavity of adaptor 180. Once the proximal end 200 of each of the second ribs 178 clears the proximal end of slot 198, the proximal ends of second pair of member 196 move back toward the longitudinal axis of the adapter. In this manner torquer 170 is secured to adaptor 180. As described above torquer nut 174 is snap fit to adaptor 180 such that manual or robotic rotation of adaptor 180 about its longitudinal axis results in the same manual or robotic rotation of torquer 170. Additionally, axial movement of adaptor 180 results in the same axial movement of torquer 170. In one embodiment a portion of second pair of ribs 178 contact both a proximal end of slot 198 and a distal end of slot 198. In one embodiment Adaptor 180 is robotically controlled in device module 148 as described above with respect to adaptor 100. Other off the shelf torquers may also be connected or secured to an adaptor by providing appropriate engagement members on the adaptor to secure the torquer axial and rotationally to the adaptor.

In one embodiment a torquer for use with certain EMDs such as a stent retriever and certain coils where it is undesirable to rotate the proximal shaft, the adaptor is not provided with a driven member. In one embodiment the adaptor includes a feature such as a tab that engages with a stop on the cassette or device module to prevent rotation of the adaptor and certain EMDs. In one embodiment driven member 190 can be located on any outer portion of the torquer body. Driven member 190 may be another type of gear such as a spur gear, worm gear, hypoid gear or could be a surface that frictionally engages a drive member including but not limited to a belt drive mechanism.

In one embodiment the driven member is located on any outer portion of the torquer body and/or may be located on an outer portion of the actuator or nut.

Although the present disclosure has been described with reference to example embodiments, persons skilled in the art will recognize that changes may be made in form and detail without departing from the spirit and scope of the defined subject matter. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described is manifestly intended to be as broad as possible. For example, unless specifically otherwise noted, the definitions reciting a single particular element also encompass a plurality of such particular elements.

What is claimed is:

1. An adaptor engaging a torquer comprising:
the adaptor including an adaptor body having a pathway therethrough includes a receptacle portion defining a cavity having an opening at a first end of the adaptor body receiving the torquer within the cavity through the opening; and
the receptacle portion including an engagement member securing the torquer to the adaptor body; the torquer being movable with the adaptor body along and about a longitudinal axis of the adaptor body.

2. The adaptor of claim 1, the torquer including a torquer body and a nut rotatable relative to the torquer body, the nut being snap fit into the cavity of the receptacle portion.

3. The adaptor of claim 2, wherein the engagement member includes at least one tab having a portion that snap fit over the nut.

4. The adaptor of claim 1, wherein the adaptor body includes a grip portion extending along a longitudinal axis of the adaptor body in a direction away from the receptacle portion and the opening of the cavity.

5. The adaptor of claim 4, the torquer including a torquer body, wherein the adaptor body and a nut is rotatable relative to the torquer body to releasably pinch an elongated medical device extending through the torquer and the pathway by rotating the grip portion relative to the torquer body.

6. The adaptor of claim 5, wherein the adaptor body includes a driven member intermediate the opening of the cavity and the grip portion.

7. The adaptor of claim 6, wherein the driven member is a gear.

8. The adaptor of claim 5, wherein the adaptor body includes an extension member portion extending from the grip portion in a direction away from the opening of the cavity, the extension member portion having a free end defining a second end of the adaptor opposite the first end.

9. The adaptor of claim 8, wherein an outer diameter of the grip portion is greater than an outer diameter of the extension member portion.

10. The adaptor of claim 4, wherein the adaptor body includes an outer bearing portion intermediate the first end and a second end of the adaptor body.

11. A robotic drive system for a catheter procedure comprising:
a device module including a drive member;
an adaptor including an adaptor body having a pathway therethrough and a receptacle portion defining a cavity having an opening at a first end of the adaptor body removably receiving a torquer within the cavity;
the receptacle portion including an engagement mechanism securing the torquer to the adaptor body; a portion of the torquer being movable with the adaptor body along and about a longitudinal axis of the adaptor body; and
the adaptor body including a driven member operatively connected to the drive member to rotate the adaptor body and torquer about a longitudinal axis of the adaptor.

12. The robotic drive system of claim 11, the torquer including a torquer body and a nut rotatable relative to the torquer body, the nut being snap fit into the cavity of receptacle portion, wherein the portion of the torquer being movable with the adaptor body is the nut.

13. The robotic drive system of claim 12, wherein the receptacle portion includes at least one tab having a portion that snap fit over the nut.

14. The robotic drive system of claim 12, wherein the adaptor body includes an outer bearing portion rotatably received to a device module bearing surface.

15. The robotic drive system of claim 11, wherein the adaptor body includes a grip portion extending along a longitudinal axis of the adaptor body in a direction away from the receptacle portion and the opening of the cavity.

16. The robotic drive system of claim 15, wherein the adaptor body is rotatable relative to a nut to releasably pinch an elongated medical device extending through the torquer and the pathway by rotating the grip portion relative to the adaptor body.

17. The robotic drive system of claim 15, wherein the adaptor body includes an extension member portion extending from the grip portion in a direction away from the opening of the cavity, the extension member portion having a free end defining a second end of the adaptor opposite the first end of the adaptor, the free end of the extension member portion being closely adjacent to a flexible track.

18. The robotic drive system of claim 17, wherein an outer diameter of the grip portion is greater than an outer diameter of the extension member portion.

19. A method for securing a torquer to a robotic drive system for a catheter procedure comprising:
providing a device module including a drive member;
providing an adaptor including an adaptor body having a pathway extending therethrough and a receptacle portion defining a cavity having an opening at a first end of the adaptor body;
attaching a torquer to the adaptor by inserting a nut of the torquer into the cavity of the adaptor through the opening at the first end of the adaptor body, while a portion of the torquer is located outside of the adaptor, wherein the nut is fixed to and movable with the adaptor body along and about a longitudinal axis of the adaptor body;
securing an elongated medical device within the torquer and extending through the pathway; and
placing the elongated medical device, adaptor and torquer within the device module.

20. The method of claim 19, wherein the adaptor includes a driven member; and
further including robotically controlling a drive member in the device module to operatively rotate driven member, adaptor, torquer and elongated medical device together.
